(19) United States
(12) Patent Application Publication
(10) Pub. No.: US 2014/0056958 A1
(43) Pub. Date: Feb. 27, 2014

(54) APPARATUS, SYSTEM, AND METHOD FOR CREATING BIOLOGICALLY PROTECTED/ENHANCED SPACES IN VIVO

(71) Applicants: Charles Knezevich, Spring Valley, CA (US); Robert D. Silvetz, Spring Valley, CA (US)

(72) Inventors: Charles Knezevich, Spring Valley, CA (US); Robert D. Silvetz, Spring Valley, CA (US)

(21) Appl. No.: 14/002,718

(22) PCT Filed: Mar. 5, 2012
(86) PCT No.: PCT/US12/27740

§ 371 Date: Oct. 30, 2013

Related U.S. Application Data
(60) Provisional application No. 61/449,612, filed on Mar. 4, 2011.

(51) Int. Cl. A61L 31/00; A61K 35/74; A61M 37/00; A61L 31/16; A61M 1/36; A61K 35/12; A61K 39/07

(52) U.S. Cl. CPC A61L 31/005; A61K 35/12; A61K 35/74; A61K 39/07; A61L 31/16; A61M 1/3679; A61M 37/00
USPC 424/422; 424/93.7; 424/93.4; 424/246.1; 210/690; 604/93.01

(57) ABSTRACT

The present invention creates a biologically protected or enhanced space in vivo in a mammal to allow the mammal's immune system and/or other biological processes to function properly in order to treat autoimmune diseases. In particular, the biologically protected/enhanced space allows biological processes to occur that can therapeutically treat Type I diabetes. The biologically enhanced/protected space can also be used to promote growth of specific cells such as insulin producing islet cells of the pancreas. Microfluidic devices can also be used to remove soluble TNF receptors and autoreactive T Cells in treating autoimmune diseases. Additionally, microfluidic devices can be used to remove blood glucose, soluble insulin receptors and insulin-like growth factor (IGF) from blood in the treatment of adult onset (Type II) diabetes.

### FIELD OF THE INVENTION

The present invention relates to medical devices, systems and methods to create a biologically enhanced or protected space in vivo in a mammal to allow the mammal's immune system and/or other biological processes to function properly in order to treat autoimmune diseases. In particular, the biologically protected/enhanced space allows biological processes to occur that can therapeutically treat Type I diabetes. The biologically enhanced/protected space can also be used to promote growth of specific cells such as insulin producing islet cells of the pancreas. Microfluidic devices can also be used to remove soluble TNF' receptors and autoreactive T Cells in treating autoimmune diseases. Additionally, microfluidic devices can be used to remove blood glucose, soluble insulin receptors and insulin-like growth factor (IGF) from blood in the treatment of adult onset (Type II) diabetes.

### BACKGROUND OF THE INVENTION

Autoimmune diseases have been on the rise in the past several decades. A common theory of the genesis of autoimmune diseases is that the immune system mistakenly targets and destroys the body's own normal cells. Specifically, defective T lymphocytes (T cells) are thought to attack and destroy the body's normal tissues and these defective T cells are said to be autoreactive T lymphocytes. Eliminating these autoreactive T cells will benefit in the treatment of autoimmune diseases.

In type I diabetes, for instance, the insulin-producing beta cells of the pancreas are targeted and destroyed by autoreactive T cells thereby compromising the body's ability to control blood sugar levels because of low insulin levels. Eventually, the body stops making insulin altogether resulting in a medical emergency and death if not treated. Type I diabetics survive on daily insulin rejections. There are estimated to be more than 1 million type I diabetics.

Two cell protein pathways arc defective in animal models of Type 1 diabetes. Those defective cell protein pathways are the (a) MHC class I and self peptide pathway and (b) TNF-alpha pathway. It has been found that TNF-alpha and TNF agonists (preferably TNF receptor 2 agonists) selectively kill autoreactive T cells responsible for beta cell destruction in diabetic models.

US Published Patent Application 20100272772, incorporated herein by reference, discloses an implant device to elicit an immune response to an antigen wherein the antigen is presented to the mammal's immune system in a protected space in vivo. The device is used to treat cancer.

### SUMMARY OF THE INVENTION

Briefly, in accordance with the present invention, a mammal is treated with an implant device to (a) elicit an immune response to an antigen wherein the antigen is presented to the mammal's immune system in a protected space in vivo or (b) allow a biological process to proceed with therapeutic results. The mammal is provided with an implant device to create a biologically enhanced or protected space in vivo in the mammal to allow the mammal's immune system and/or other biological processes to function properly in order to treat autoimmune diseases.

In a broad aspect, the present invention creates an immunologically protected/enhanced space in vivo in a mammal by allowing mammalian cells, bacterial cells and/or growth factors to incubate in a defined space within the body. Placing desirable mammalian cells and/or progenitor cells and growth factors within the defined protected space allows the mammalian cells to grow and produce desirable therapeutic proteins thereby providing an artificial organ. For example, progenitor cells that will mature into insulin producing pancreatic beta cells are inserted into the protected/enhanced space along with growth factors to provide an artificial pancreas for Type 1 diabetics. In another broad aspect of the present invention, bacteria, including pathogens or microorganisms that produce antigenic determinants, can also be inserted into the protected/enhanced space to elicit an immune response that will have therapeutic effects especially therapeutic effects in the treatment of autoimmune diseases. For example, the protected/enhanced space can be seeded with Bacillus calmette Guerin to induce a TNF-alpha response. TNF-alpha is known to selectively kill autoreactive I cells that are responsible for causing Type 1 diabetes, ie, autoreactive T cells destroy pancreatic beta cells. The protected/enhanced space is created by providing defined spaces within a medical implant device which will contain desired cells and/or contain immobilized enzymes to catalyze specific reactions.

The exact configuration of the medical implant is not critical to the practice of the present invention. In one embodiment the medical implant device of the present invention contains (a) a biocompatible outer case; and (b) a biologically protected/enhanced inner space that contains (i) mammalian cells, (ii) bacterial cells and/or (iii) growth factors or other enzymes Growth factors are preferably immobilized on a solid support. The size of the implant is not critical to the practice of the present invention and the implant can be from several centimeters in length that can be implanted using a minor surgical incision to a miniaturized pellet that can be inserted with, for example, a 12 or 16 gauge needle and syringe. Preferably, the medical implant device for mammals contains a biocompatible outer case that allows flow of blood/plasma into the implant device when implanted into a mammal.

In another aspect, the present invention relates to a medical implant device used to treat Type 1 diabetes in a human wherein the implant contains (a) a porous outer biocompatible case, (b) a peripheral region within the biocompatible case that contains growth factors that stimulate the growth and maturation of progenitor cells of insulin producing pancreatic beta cells and (c) an inner region within which contains progenitor cells that will mature into insulin producing pancreatic beta cells. The peripheral region and the inner region are separated by a membrane that is porous to sub-cellular components such as antigenic proteins, inactivated viruses, cytokines, cytokine receptors and the like.

Of particular interest in practicing the present invention human patients are treated with the present medical implant device to treat an autoimmune disease. The patient is provided with an implant device that contains a protective biologically active space to (a) elicit an immune response to an antigen or (b) allow a biological process to proceed with therapeutic results. A sample of the mammalian cells or bacterial cells are placed into the innermost portion of the protective space of the implant device in a porous chamber that is porous to sub-cellular components but impervious to cells. Enzymes or growth factors can then be loaded when desired into the medical device but outside the cell-containing innermost chamber. The loaded implant device is implanted into the patient allowing an immune response or biological process to proceed within the protective space.

In one embodiment of the present invention, the implant device is made up of three concentric biocompatible hollow fiber membranes, approximately 5 cm long with an outside diameter of about 5-20 millimeters and which can be implanted with a small incision. In another embodiment the implant device comprises injectable pellets having a diameter of from about 0.5 mm to about 2 mm and a length of from about 3-10 mm or more and which can be implanted with a needle and syringe. The injectable pellets can also be made with a configuration of 3 concentric hollow fiber membranes.

The exact size and shape of the implant device is not critical to the practice of the present invention. The innermost portion of the device is seeded with mammalian cells or bacterial cells as the case may be. The outermost portions of the biologically protective space may contain growth factors.

In another aspect of the present invention a microfluidic device is used to subtract or remove elements from the blood in the treatment of autoimmune diseases. In particular, autoreactive T lymphocytes can be selectively separated from blood and destroyed by apoptosis inducing cytokines or by mechanical means. In the case of Type 2 diabetes (adult onset) soluble insulin receptors, insulin like growth factor (IGF) and glucose are removed from blood passed through a microfluidic device.

The various medical implant devices and associated methods of the present invention create a biologically protected/enhanced space in vivo.

### BRIEF DESCRIPTION OF THE DRAWINGS

FIG. 1 is a cross sectional view of a medical implant device of the present invention configured as 2 concentric hollow fiber membranes.

### DETAILED DESCRIPTION OF THE INVENTION

In practicing the present invention, a mammal is treated with a subcutaneous implant device to elicit an immune response to an antigen wherein the antigen is presented to the mammal's immune system in a protected space in vivo or to allow a biological process to proceed that has therapeutic effects. The mammal can be any mammal but humans, non-human primates, dogs, cats, horses, zoo animals, prized breeding bulls and any other commercially valuable agricultural animals are preferred. Any description herein relating to humans is equally applicable to other mammals.

The following definitions apply to the practice of the present invention:

The term “autoimmune disease” refers to any disease where the immune system mistakenly attacks the body's own tissues and cells. Type 1 diabetes is an autoimmune disease in, which the insulin-producing beta cells of the pancreas are the target of the immune attack. Other autoimmune diseases include lupus, Crohn's disease, multiple sclerosis, scleroderma, Sjogren's syndrome, and rheumatoid arthritis.

The term “Type 1 diabetes” means” a dysfunction in the regulation of blood glucose levels from reduced insulin level due to damage to insulin-producing beta cells by the immune system of the host. The reduction in insulin is usually quick and usually results in the total lack of insulin production.

The term “Type 2 diabetes” and “adult onset diabetes” refers to a disease of energy dysfunction due in part to insulin resistance.

The mammal is provided with an implant device that contains a protective biological space to elicit an immune response to an antigen wherein the antigen is presented to the mammal's immune system in a protected space in vivo or to allow a biological process to proceed that has therapeutic effects One or more mammalian cells or bacterial cells are placed into the innermost portion or chamber of the protective space of the implant device. This innermost chamber contains the cells within the innermost portion of the protective space and prevents cellular contact with other cells in the protective space and release of the mammalian or bacterial cells into the host mammal. However, the walls of the chamber are pervious to sub-cellular components contained within the protective space. The loaded implant device is implanted into the mammal allowing an immune response or a biological process to proceed within the protective space that provides a therapeutic effect in the treatment of an autoimmune disease such as Type 1 diabetes.

The implant device of the present invention is made with known biocompatible polymers, biopolymers or composite material porous membranes employing manufacturing procedures well known to one of ordinary skill in the art. Polymer membranes having a pore size of from about 0.6 to about 60 μm or more are advantageous. Preferably, the inner membranes (described below) have a pore size less than about 1 μm and the outer membrane (described below) less than about 5 μm. The outermost membrane should promote vascularization around the outside of the implant and membranes having a pore size of about 60 μm are preferred for the purpose of promoting vascularization. All of the membranes should allow sub-cellular components to move freely through the membrane. Suitable polymer membranes include mixed esters of cellulose having a nominal pore size ranging from 1.2 to 8.0 μm; cellulose acetate having a nominal pore size ranging from 0.8 to 8.0 μm; and PTFE/polyester having a nominal pore size ranging from 1.0 to 15 μm. See U.S. Pat. No. 5,964,804 which is incorporated herein by reference.

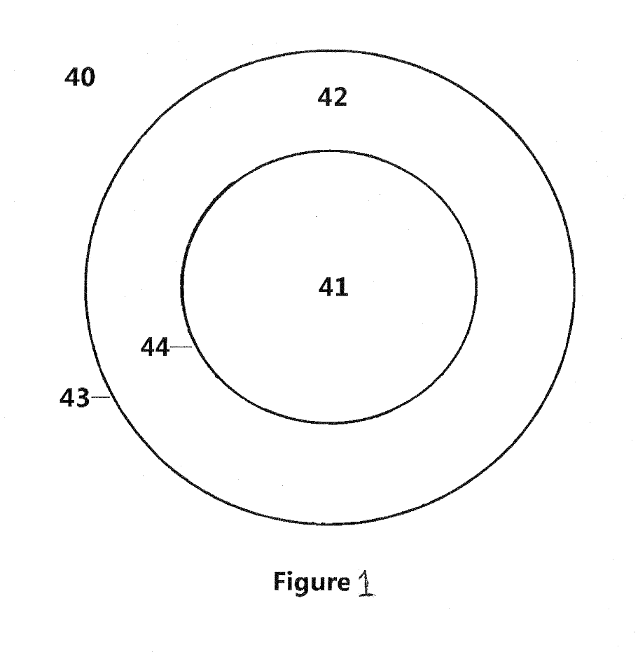

The innermost chamber of the medical implant device that contains the mammalian or bacterial cells preferably contains a diaphragm through which the antigens are injected into the cell-containing chamber and contained therein. The diaphragm prevents escape of the cells into the patient's circulatory system. For example the diaphragm will form the end of the inner antigen chamber/compartment 41 shown in FIG. 1. The diaphragm is attached to the end of the medical implant device employing standard manufacturing techniques. The chambers containing the cells are loaded by injecting the cells through the diaphragm and into the chamber.

A preferred configuration of the present medical implant device has two concentric hollow fiber membranes having (a) mammalian cells or bacterial cells contained in the innermost hollow fiber space and (b) one or more cell growth factors in the outer hollow fiber space when growth factors are desired to stimulate the growth and maturation of cells contained in the innermost hollow fiber space.

Mammalian cells and bacterial cells that are added to the present implant can be any cells to which an immune response by the patient is desired or which produces a therapeutic compound, such as, for example, insulin. In one embodiment progenitor cells that will mature into pancreatic insulin producing beta cells are added to the implant device along with growth factors that stimulate the growth and maturation of the progenitor cells. The implant is then surgically inserted subcutaneously. Once the progenitor cells have matured into insulin producing cells they will express insulin in response to blood glucose fluctuations and regulate blood glucose levels similar to the beta cells in a non-diabetic individual. In this respect the present medical implant serves as an artificial pancreas.

In another embodiment Bacillus calmette Guerin cells are added to the implant device so that the bacteria will stay positioned within the implant. The Bacillus calmette Guerin arc employed to induce a TNF-alpha response with the goal of killing autoreactive T cells that are responsible for destroying pancreatic beta cells. Preferably, when Bacillus calmette Guerin cells are inserted into the medical implant the patient is subjected to a microfluidic process to remove soluble TNF receptors from the patient which will amplify the TNF-alpha wave response. The cells are placed in an inner compartment of the present implant device wherein the compartment wall is porous to sub-cellular components but impervious to cells. It is important that bacteria and mammallian cells are contained within the inner compartment and not released into the blood stream of the mammal being treated. Bacteria cells or mammalian cells are harvested and placed into the inner compartment which is then sealed to prevent release of the cells into the mammal's blood stream. Alternatively, the present implant device is made having a diaphragm that allows the bacterial cells or mammalian cells to be injected directly into the inner, cell-containing compartment.

Optionally, abrasive materials can be added to the inner cell-containing compartment. The abrasive materials will control overgrowth of cells in the inner compartment when implanted into a mammal. The abrasive materials mechanically disrupt cell wall membranes and will control overgrowth of bacterial or mammalian cells contained within the inner compartment while the implant is implanted within a mammals' body. The abrasive material can be abrasive heads or abrasive fibers. Abrasive beads can be coated with abrasive inorganic compounds. Suitable abrasive fibers include silicon fiber whiskers and ceramic whiskers.

In another embodiment a microfluidic device is employed to channel T cells by size into a chamber that binds and separates autoreactive T cells from the flow of normal T cells. The captured autoreactive T cells are destroyed by exposure to apoptosis inducing cytokines or mechanical means where the cell membranes are disrupted. The T cells are separated by binding with antibodies or other polyclonal means. Over a 3-6 months period billions of autoreactive T cells can be killed resulting in the decreased destruction of normal cells, ie in the case of Type 1 diabetes there would be less damage occurring to normal pancreatic beta cells. This continued drain on the production of autoreactive T cells will eventually result in clonal exhaustion at the stem cell level and elimination of the autoreactive clone from circulation. Elimination of the autoreactive clone will stop the attack of the normal cells and cessation of the autoimmune disease. In the case of treating Type 1 diabetes since the pancreas sits in the portal spleen circulation regeneration of the beta islet cells will occur. This microfluidic process of autoreactive T cell removal can be used in combination with the medical implant embodiments described herein. For example, the autoreactive T cells responsible for Type 1 diabetes can be removed by the microfluidic process in combination with medical implants that contain Bacillus calmette Guerin cells and/or medical implants that contain progenitor cells and growth factors to stimulate the production of insulin producing beta cells.

In a further embodiment of the present invention Type 2 diabetes, although not usually considered an autoimmune disease, is treated with a microfluidic process where soluble insulin receptors and IGF are removed to improve insulin sensitivity, ie, reduce insulin resistance. Additionally, glucose can also he microfluidically removed from blood to further decrease blood glucose levels. The removed glucose can be shunted to a cartridge reservoir and changed on a regular basis.

The implant is inserted into any subcutaneous tissue in the body. Suitable locations include the inner aspect of an arm, the lateral midline area of the chest below the arm pit, a side region of the abdomen and the inner aspect of the thigh. The implant insertion can conveniently be done as a minor surgical procedure in an outpatient facility. Additional implants can be administered in different body locations as deemed necessary by a healthcare practitioner. As one implant is removed a new implant can be administered. Alternatively, additional implants are spaced out at one or 2 week intervals. In another embodiment, two or more implants can be implanted at the same time. The patient's autoimmune disease is monitored using standard diagnostic/monitoring techniques such as blood testing to determine blood glucose levels in the case of a diabetic or other suitable markers in a patient with a different autoimmune disease.

The medical implant device of the present invention can be administered to a patient at anytime but it is preferred to administer the implant when the patient's immune system is not in a compromised condition such as during and shortly after the administration of chemotherapy or radiation treatments. Once the patient's white blood count is within normal ranges then the present implant is preferably administered. Other lifestyle choices to bolster the patient's immune system are of benefit. These lifestyle choices include plenty of sleep, moderate exercise, an insulin lowering diet, consumption of EPA and DHA as well as other omega-3 fatty acids such as those found in cold water ocean fish (salmon, tuna, sardines, mackerel, etc)., a reduction in omega-6 fatty acids, elimination of trans-fats from the diet, restricted ingestion of alpha linolenic acid and the like. Immune enhancing supplements, such as Vit C, co-enzyme Q-10 and Vit D, can also be taken.

In a preferred embodiment of the present invention, the loaded implant is placed into the patient by inserting it into a small incision made at a desirable location or by injecting implant pellets with a needle and syringe. The implant is left in the patient's body so long as a therapeutic effect is achieved.

FIG. 1 is a drawing that shows a cross-sectional view of an implant of the present invention 40 used to treat diabetes and is made of 2 concentric hollow fiber membranes 43 and 44. The innermost hollow fiber membrane 43 defines an inner antigen chamber or compartment 41 which contains progenitor pancreatic beta cells (not shown). Hollow fiber membrane 43 is impervious to cells but pervious to sub-cellular components such as antigens, cytokines, cytokine receptors, and the like. Hollow fiber membrane 43 is sealed at both ends to prevent cancer cells (not shown) from being released into the patient's vascular system. The outer hollow fiber membrane 44 defines the outer chamber or compartment 42 which contains a one or more growth factors (not shown) that will stimulate the progenitor cells grow and mature into insulin producing pancreatic beta cells. The outer hollow fiber membrane 44 is impervious to cells but pervious to sub-cellular components such as antigens, cytokines, cytokine receptors, and the like. Hollow fiber membrane 44 is sealed at both ends. Hollow fiber membrane 44 also promotes vascularization and inhibits encapsulation by the patient's immune system.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A method of creating a biologically protected/enhanced space in vivo in a mammal which comprises:
a. seeding a defined space within the mammal's body with desirable mammalian or bacterial cells and
b. allowing the cells to grow to produce a therapeutic response.

2. The method of claim 1 further comprising seeding the defined space with a growth factor that stimulates the growth of the mammalian or bacterial cells.

3. The method of claim 2 wherein the defined space is seeded with progenitor cells to insulin producing pancreatic beta islet cells and one or more growth factors that will stimulate the progenitor cells to mature into insulin producing beta cells and the mammal has Type 1 diabetes.

4. The method of claim 1 wherein the defined space is seeded with Bacillus calmette Guerin to induce a TNF-alpha response and the mammal has Type 1 diabetes.

5. The method of claim 1 wherein the defined space is seeded with more than one bacterial species to colonize said bacterial species to induce a therapeutic response.

6. The method of claim 1 wherein the mammal is a human that has an autoimmune disease.

7. The method according to claim 1 wherein the mammal has Type 1 diabetes and the method comprises:
a. providing an enclosed space within the mammal, said enclosed space comprising an internal region and a peripheral region wherein the peripheral region contains a pancreatic beta cell growth factor;
b. placing within the internal region of the enclosed space progenitor cells of pancreatic insulin producing beta cells; and
c. allowing the progenitior cells to mature into pancreatic insulin producing beta cells wherein said mature beta cells produce insulin in response to blood sugar elevations.

8. The method according to claim 1, wherein the mammal has Type 1 diabetes and the method comprises:
a. providing an enclosed space within a mammal, said enclosed space comprising an internal region and a peripheral region wherein the peripheral region or the internal region is seeded with Bacillus calmette Guerin; and
b. allowing the Bacillus calmette Guerin cells to grow and induce a TNF-alpha response to selectively kill autoreactive T cells that are responsible for causing Type 1 diabetes.

9. A method of treating a mammal that has an autoimmune disease with an implant device which comprises:
a. providing an implant device that contains a protective space for mammalian or bacterial cells wherein said protective space contains the cells within the implant device;
b. placing one or more mammalian or bacterial cells into the protective space of the implant device;
c. inserting the loaded implant device into the mammal; and
d. allowing the cells to incubate and produce a therapeutic effect in the treatment of the autoimmune disease.

10. The method of claim 9 wherein the mammal is a human, the cells are Bacillus calmette Guerin cells and the autoimmune disease is Type 1 diabetes.

11. The method of claim 9 wherein the mammal is a human, the cells are progenitor cells of pancreatic insulin-producing beta cells, the autoimmune disease is Type 1 diabetes and wherein the device further comprising one or more growth factors that stimulate the progenitor cells to grow and mature into insulin producing pancreatic beta cells.

12. The method of claim 10 further comprising microfluidically removing soluble TNF receptors and/or autoreactive T-cells from the human.

13. (canceled)

14. A method of treating a Type 2 diabetes patient which comprises microfluidically removing soluble insulin receptors and insulin-like growth factor (IGF) from the patient's blood.

15. A medical implant device for a patient with an autoimmune disease which comprises:
a. a porous biocompatible outer case and
b. a biologically protected/enhanced inner space that contains one or more mammalian or bacterial cells wherein said cells are contained within the inner space and cannot enter the patient's vascular system.

16. The medical implant device of claim 15 wherein the cells are Bacillus calmette Guerin cells and the autoimmune disease is Type 1 diabetes.

17. The medical implant device of claim 15 wherein the cells are progenitor cells of pancreatic insulin-producing beta cells, the autoimmune disease is Type 1 diabetes and the device further comprising one or more growth factors that stimulate the progenitor cells to grow and mature into insulin producing pancreatic beta cells.

18. The medical implant device of claim 15 wherein the cells are more than one bacterial species wherein said species colonize to induce a therapeutic response.

19. The medical implant device of claim 15 wherein the porous biocompatible outer case comprises polymeric components that are impervious to cells but porous to sub-cellular components.

20. The medical implant device of claim 9 wherein the implant device is administered by insertion through a small incision in the skin into subcutaneous tissue.

21. (canceled)

22. A medical implant device to produce a therapeutic response in a patient with an autoimmune disease which comprises:
a. a porous outer biocompatible case,
b. a peripheral region within the biocompatible case that contains a growth factor, and
c. an inner region which contains mammalian cells
wherein the peripheral region and the inner region are separated by a membrane that is porous to sub-cellular components but not to cells.

23. The medical implant device of claim 22 wherein the mammalian cells are progenitor cells of pancreatic insulin-producing beta cells, the growth factor is a growth factor that stimulates the progenitor cells to grow and mature into insulin producing pancreatic beta cells and the autoimmune disease is Type 1 diabetes.

24. A medical implant device for a patient with an autoimmune disease which comprises:
a. a biocompatible outer case that allows flow of blood into the implant device when implanted into the patient and
b. a biologically protected/enhanced inner space which contains mammalian cells or bacterial cells
whereby the cells grow when implanted into the patient and produce a therapeutic effect.

25. The medical implant device of claim 24 wherein the therapeutic effect is the killing of autoreactive T cells in the patient.

26. The medical implant device of claim 25 wherein the autoreactive T cells are the T cells responsible for causing type 1 diabetes.

27. The medical implant device of claim 24 wherein the therapeutic effect is achieved by the expression of a protein from the cells.

28. The medical implant device of claim 27 wherein the protein is insulin.

29. The device according to claim 22 which comprises:
a. a porous outer biocompatible case,
b. a peripheral region within the biocompatible case that contains a growth factor that stimulates the growth and maturation of progenitor cells of pancreatic insulin-producing beta cells, and
c. an inner region which contains progenitor cells of pancreatic insulin-producing beta cells
wherein the peripheral region and the inner region are separated by a membrane that is porous to sub-cellular components but not to cells.
